(19) United States
(12) Patent Application Publication
(10) Pub. No.: US 2015/0359993 A1
(43) Pub. Date: Dec. 17, 2015

(54) Bathroom Performance System

(71) Applicant: Matthew Samar, San Diego, CA (US)

(72) Inventor: Matthew Samar, San Diego, CA (US)

(21) Appl. No.: 14/657,464

(22) Filed: Mar. 13, 2015

Related U.S. Application Data
(60) Provisional application No. 62/011,759, filed on Jun. 13, 2014.

(51) Int. Cl. A61M 21/02; G06F 17/30; H03G 3/20; A47K 17/02

(52) U.S. Cl. CPC A61M 21/02; A47K 17/02; G06F 17/3074; H03G 3/20; A61M 2021/0027

(57) ABSTRACT

The present invention is a device removably mounted on or within the vicinity of a bathroom toilet configured to assist paruretics with performance and a method for using the same. The device includes a housing enclosing a detection assembly therein having a motion sensor, a microprocessor, and a speaker system having a digital-to-analog converter, an amplifier and a speaker. The motion sensor is configured to detect a presence of a user within a general vicinity, wherein detection of the presence of a user prompts the motion sensor to transmit a digital signal to the microprocessor. Upon receipt of the digital signal, the microprocessor is configured to retrieve a digital recording file from a computer storage and transmit the digital recording file to the speaker system to drive the speaker. The digital recording file includes a continuous sound to alleviate stress of a user and promote bathroom performance.

### CROSS REFERENCE TO RELATED APPLICATION

This application claims the benefit of U.S. Provisional Application No. 62/011,659 filed on Jun. 13, 2014. The above identified patent application is herein incorporated by reference in its entirety to provide continuity of disclosure.

### BACKGROUND OF THE INVENTION

### 1. Field of the Invention

The present invention relates to bathroom accessories. More specifically, the present invention pertains to an improved detection assembly removably mounted on or within the vicinity of a toilet tank or urinal to assist paruretics with performance. The present invention includes a housing that holds a detection assembly having a motion sensor, a microprocessor, and a speaker system.

Difficulty urinating in the presence of others is called Paruresis. Often referred to as “shy” bladder” syndrome, Paruresis is a phobia that involves fear and avoidance of using public bathrooms and an irregular idiopathic form of urinary retention. This form of urinary retention is not caused by a physical blockage or function in the body. People who suffer from Paruresis also have trouble urinating in the presence of others. More specifically, Paruresis is the fear of not being able to urinate with some or complete privacy. The severity of the symptoms depends on the level of fear and stress. Paruresis can interfere drastically in a person's quality of life. For example, a paruretic may have difficultly when ordered to submit a urine analysis on command for drug testing in a public facility. Other difficulties range from traveling on long plane rides to every day social activities and situations.

There can be many causes to Paruresis. Some paruretics are afflicted at a very young age, however a person of any age can be afflicted with the disorder. Some paruretics can point to a specific triggering incident that was traumatic. These triggering events incidents include, but are not limited to, embarrassment by a parent, teasing by classmates or siblings. Other paruretics also cannot point to any specific triggering event. Paruresis can eventually lead to health problems, such as an enlarged prostate, kidney stones, bladder infections and other ailments.

A typical paruretic is able to anticipate difficulty urinating whenever entering a bathroom. Attempts to manually control the process are unsuccessful, and the associated anxiety with performance greatly reduces the individual's chance of urinating while in a public facility. The paruretic then reacts, by allowing the disorder to control their habits. Some of these developed habits include urinating as much as possible at home, restricting the intake of fluids, locating private and vacant public bathrooms, or refusing extended social invitations to avoid urinating in a facility other than their own. Therefore, there exists a need for a device that alleviates stress and promotes performance for a paruretic

The present invention provides a solution with a device removably mounted on or within the vicinity of a toilet tank or urinal adapted to assist paruretics with performance in a public facility such as ones in a restaurant, school, and commercial buildings. The present invention includes a housing that holds a detection assembly having a motion sensor, a microprocessor, and a speaker system. The motion sensor detects the presence of a user and triggers the transmission of a signal to the microprocessor. The microprocessor, upon receipt of the signal, retrieves a digital recording file from a computer storage and transmits the digital recording file to the speaker system. The digital recording file includes a continuous sound having a repeating loop of noise, such as the sound of running water, designed to alleviate stress of a user and promote bathroom performance. The motion sensor is also designed to detect the departure of a user, which stops the release of the audio recording. In this way, the audio recording is configured to mask the sound or urinating, thereby saving the user from embarrassment, which may cause stress and decrease bathroom performance.

2. Description of the Prior Art

Devices have been disclosed in the prior art that relate to motion sensor systems for toilets. These include devices that have been patented and published in patent application publications. Some of these devices provide a method of requesting assistance for a patient. Other devices provide a flushing system adapted to electronically activate a flushing valve on a toilet. These devices, however, do not provide a device that is configured to detect a presence near the general vicinity of a toilet activating a digital recording file comprising a continuous sound having a repeated loop of noise designed to alleviate stress of a user, such as a paruretic, and promote bathroom performance. The foregoing is a list of devices deemed most relevant to the present disclosure, which are herein described for the purposes of highlighting and differentiating the unique aspects of the present invention, and further highlighting the drawbacks existing in the prior art.

Specifically, U.S. Pat. No. 8,461,988 to Tran provides a method of requesting assistance for a patient. The Tran device a patient-worn wireless device with at least one accelerometer to detect motion. The wireless device further includes a button for manually transmission of an assistance request. The Tran device is adapted to extract an initial set of signature body motions and compare it to daily activities to identify a fall motion. In response, the Tran device then automatically requests assistance using a telephone for the patient. However, the Tran device does not disclose a speaker system activated by a motion sensor that plays a continuous sound comprising a repeating loop of noise such as running water.

Similarly, U.S. Pat. No. 7,797,769 to Ozenick provides a flushing system adapted to electronically activate a flushing valve on a toilet. The Ozenick device provides a pair of motion sensors on opposite sides of a toilet responsively coupled to the electronically activated flushing valve. The Ozenick device further provides a sensing module having a control circuit having a detector that triggers a sub-circuit, wherein the sensing module is coupled with the flushing valve. However, the Ozenick device does not provide a system for promoting bathroom performance having a speaker system.

Published U.S. Patent Application No. 2012/0068613 to Veneto provides a light assembly for a toilet, wherein if a user walks in a bathroom near a toilet, the motion detector will sense their presence and automatically turn on the bathroom lights. The Veneto device provides a motion detector configure to sense a presence that is coupled with a control circuit. The control circuit is also coupled with the bathroom lights and configured to activate the lights in response to the motion detector. However, the Veneto device provides a light assembly for a toilet, and does not provide a system for promoting bathroom performance having a speaker system configured to play a repeating loop to alleviate stress of a user and promote bathroom performance.

U.S. Patent No. 2003/0154541 to Miller provides a device for automatically raising and lowering a toilet seat. The Miller device provides a housing consisting if a sensor, a motor and a spool that is removably attached to a toilet tank. The sensor is coupled to the motor, wherein the sensor sends an activation signal to the motor upon detection of motion. The motor is then triggered to rotate the spool which includes a line that is adapted to raise and lower a toilet seat. However, the Miller device provides a device for raising and lower a seat and does not provide a speaker system activated by a motion sensor.

Finally, Published U.S. Patent Application No. 2012/0317709 to Oates provides an automatic flush apparatus to convert a manual flushing toilet to an automatic one. The Oates device provides a sensor mounted on the exterior wall of a tank adapted to detect a predetermined condition. The Oates device further contains a signal transmitter that conveys a signal indicating the occurrence of the condition and a motor operatively coupled to the flush valve adapted to initiate the flush valve upon receipt of a signal. However, the Oates device provides a device for automatically flushing a toilet and does not provide a speaker system configured to play a continuous sound comprising a repeating loop of noise, such as running water.

The devices disclosed in the prior art have several known drawbacks. These devices are limited to devices that disclose methods for automatically requesting assistance for a patient, turning on bathroom lights, raising a toilet seat, or flushing a toilet. The present invention overcomes their limitations by disclosing a device that detects a presence near the general vicinity of a toilet, which activates a speaker system to play a repeating loop of noise designed to alleviate stress of a user, such as one that is afflicted with Paruresis, and promote bathroom performance.

The present invention comprises a housing that holds a detection assembly having a motion sensor, a microprocessor, and a speaker system. The motion sensor detects the presence of a user within a general vicinity of a toilet and transmits an output digital signal to the microprocessor indicating the presence of a user. The microprocessor, in receipt of the signal from the motion sensor, retrieves a digital recording file from a computer storage and transmits the same to the speaker system. The digital recording file comprises a continuous sound having a repeating loop of noise that alleviates the stress of a user and promotes bathroom performance. It is therefore submitted that the present invention is substantially divergent in design elements from the prior art, and consequently it is clear that there is a need in the art for an improvement to motion sensor systems for toilets. In this regard, the instant invention substantially fulfills these needs.

### SUMMARY OF THE INVENTION

In view of the foregoing disadvantages inherent in the known types of bathroom performance systems now present in the prior art, the present invention provides a new and improved bathroom performance system adapted to detect a presence near a general vicinity of a toilet and transmit a signal to activate a speaker system that is configured to play a continuous sound to alleviate stress of a user and promote bathroom performance.

It is therefore an object of the invention to provide a new and improved bathroom performance system that has all of the advantages of the prior art and none of the disadvantages.

Another object of the present invention is to provide a new and improved bathroom performance system that detects a user's presence near a general vicinity of a toilet and transmits an output digital signal to activate a speaker system to play a continuous sound to alleviate stress of a user and promote bathroom performance.

Yet another object of the present invention is to provide a new and improved bathroom performance system that comprises a housing storing a detection assembly therein comprising a motion sensor, a microprocessor, and a speaker system having a digital-to-analog converter, an amplifier, and a speaker.

Still yet another object of the present invention is to provide a new and improved bathroom performance system that is removably mounted to a toilet tank, urinal, or the general vicinity of either fixture.

A further object of the present invention is to provide a new and improved bathroom performance system, wherein the continuous sound is a repeating loop of noise, such as running water that is configured to assuage anxiety of a paruretic caused by the presence of others in a public setting that results in an inability to perform.

Another object of the present invention is to provide a new and improved bathroom performance system, wherein the audio recording is configured to mask the sound or urinating, thereby saving the user from embarrassment, which may cause stress and decrease bathroom performance.

Still yet another object of the present invention is to provide a new and improved bathroom performance system wherein the device may be readily fabricated from materials that permit relative economy and are commensurate with durability.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein the numeral annotations are provided throughout.

FIG. 1 shows a schematic diagram of the present invention.

FIG. 2 shows the present invention as removably mounted on a wall near a urinal.

### DETAILED DESCRIPTION OF THE INVENTION

References are made herein to the attached drawings. Like reference numerals are used throughout the drawings to depict like or similar elements of the bathroom performance system. For the purposes of presenting a brief and clear description of the present invention, the preferred embodiment will be discussed as removably mounted on a toilet tank, urinal, or within the vicinity of either fixture. The figures are intended for representative purposes only and should not be considered to be limiting in any respect.

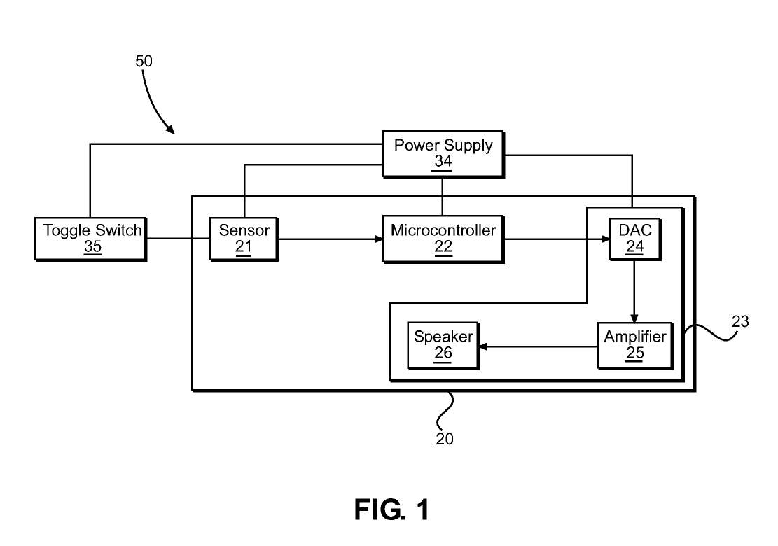

Referring now to FIG. 1, there is shown a schematic diagram of the detection assembly 20 of the bathroom performance system 50. The bathroom performance system 50 is removably mounted on a toilet tank, a urinal or within a general vicinity of either fixture adapted to assist paruretics with bathroom performance. The bathroom performance system 50 comprises a housing that stores a detection assembly 20 having a motion sensor 21, a microprocessor 22, and a speaker system 23. The speaker system 23 further comprises a digital-to-analog converter (DAC) 24, an amplifier 25, and a speaker 26. The motion sensor 21 detects presence of a user within a general vicinity and transmits a digital output signal to the microprocessor 22. The microprocessor 22, which is also coupled with a computer storage 27, retrieves a digital recording file from the computer storage 27 and transmits the digital recording file to the speaker system 23 to drive the speaker 26. The computer storage 27 is preferably a Random Access Memory (RAM) or a hard drive, however, other suitable alternatives are used in other embodiments.

The digital recording file transmitted from the microprocessor 22 is converted into an analog signal by the DAC 24. The analog signal is then amplified by the amplifier 26 and the digital recording file, now in an analog signal, is played through the speaker 26. The motion sensor 21 also detects when the user steps out of the general vicinity of the motion sensor 21, thereby deactivating the speaker system 23.

The motion sensor 21 may rely on any means, including microwave energy, ultrasonic waves, or radio frequency signals, to detect a user approaching a toilet, however, infrared means are preferred. In the preferred embodiment, the motion sensor 21 is a passive infrared detector (PIR). The motion sensor 21 is sensitive to changes in radiation from within the range. A user within the range may emit infrared radiation of sufficient magnitude to trigger the motion sensor 21 to provide an output digital signal.

Appropriate threshold conditions for triggering motion sensor 21 may also be established. For example, the motion sensor 21 may be configured to provide an output digital signal only when movement of a source of radiation above threshold speed or threshold amount is detected. Moreover, the range of the motion sensor 21 may be narrowly focused to avoid detecting extraneous movements. Too large of a range will unnecessarily activate the speaker system 23 and too small a coverage area will not activate the speaker system 23 when required. When the user leaves the range, appropriate threshold conditions for triggering the motion sensor 21 are not established, and motion sensor 21 does not provide an output digital signal. Preferably, the motion sensor 21 is triggered to transmit a digital output signal when a user reaches a general vicinity to a bathroom toilet or a urinal.

The digital output signal is transmitted to the microprocessor 22. The microprocessor 22 does not have to be sophisticated as it functions primarily as a pathway between the motion sensor 21 and the DAC 24 of the speaker system 23, however the microprocessor 22 is also configured to retrieve the digital recording file stored on the computer storage 27. The digital recording file is transmitted to the DAC 24, which primarily functions to convert the digital recording file retrieved by the microprocessor 22 to an analog signal to drive the speaker 26. The amplifier 25 intensifies generally low powered analog signals from the DAC 24 to a level suitable for driving speaker 26. Generally, an input signal to the amplifier 25 may only be a few hundred microwatts, however, the amplifier 25 can output tens to hundreds of watts based on preference. The speaker 26 receives the output analog signal from the amplifier 25 and plays a continuous sound original stored as the digital recording file in the computer storage 27.

The digital recording file stored in the computer storage 27 is a continuous sound that comprises a repeating loop of noise. The continuous sound is configured to alleviate stress of a potential bathroom user and promote bathroom performance. In this way, the continuous sound is particularly advantageous for bathroom users that suffer from Paruresis as it masks the sound of urinating, which saves the bathroom user much embarrassment, especially in a public restroom with other people nearby. The repeating loop of noise includes sound of running water, white noise, and any other suitable sounds that may help people with Paruresis.

The motion sensor 21 is also electrically coupled to an on/off toggle switch 35 that must always be turned on for the detection assembly 50 to operate. Preferably, the toggle switch 35 is a standard toggle switch, however, other embodiments are alternatively composed of other suitable types of switches like rocker switches. In addition, the detection assembly 50 and the on/off toggle switch 35 is electrically coupled to a power supply 34. Specifically, the power supply 34 is coupled to the motion sensor 21, the microprocessor 22 and the speaker system 23 of the detection assembly 50. Preferably, the power supply 34 is a battery, such as alkaline batteries or rechargeable batteries, however, other embodiments may be alternatively composed of other suitable means for power, such as a power cord plugged into a standard electrical outlet.

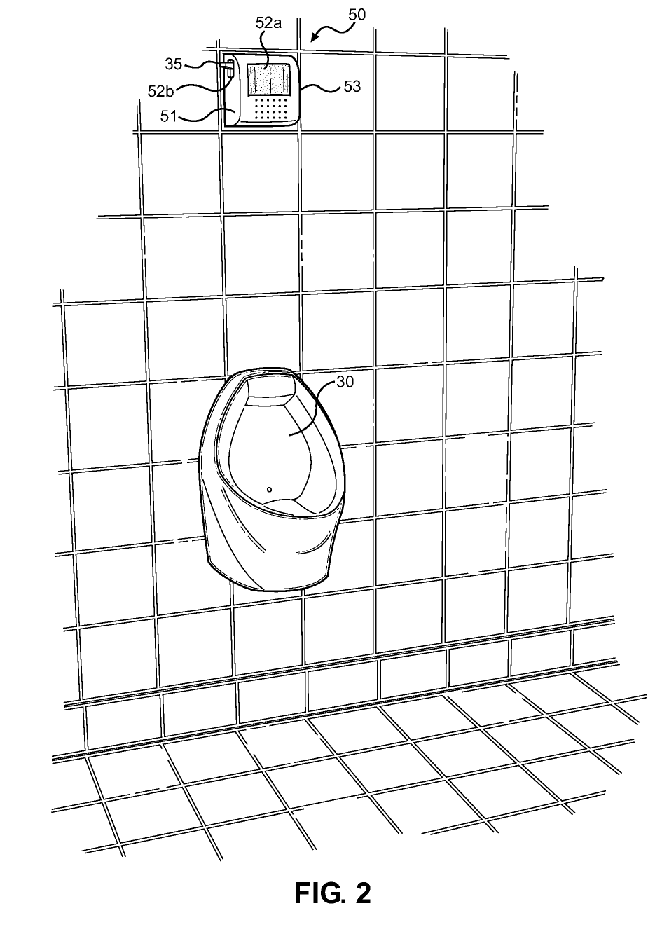

Referring now to FIG. 2, there is shown a view of the bathroom performance system 50 as removably mounted within the vicinity of a urinal 30. The bathroom performance system 50 comprises a housing 51 that receives and stores the detection assembly 20 therein. The housing 51 comprises one or more sides 53 enclosing an interior chamber utilized to receive and store the detection assembly 20 therein. The housing 51 further comprises a rear surface with an adhesive that lays plush against and removably attaches to a wall or other suitable surfaces in a general vicinity of a toilet or a urinal. Without limitation, other embodiments comprise other suitable fasteners such as pressure sensitive tape, and varying shapes and sizes for housing 51. The side 53 further includes a pair of openings 52A, 52B, wherein the first opening 52A is configured to provide access to the on/off toggle switch 35, and the second opening 52B is configured to provide a line of sight for the motion sensor. In another preferred embodiment, the housing 51 is configured to be removably attached to an upper side of a urinal flush valve.

The on/off toggle switch 35 is configured to deactivate the motion sensor to facilitate any adjustments, such as moving the bathroom performance system 50 to another location or the changing of a battery. In addition, preferably, the bathroom performance system 50 comprises a passive infrared sensor, which conventionally requires a direct line of sight between the motion sensor and the general vicinity around the motion sensor.

In alternative embodiments, the existing sensor on an automatic flushing toilet or urinal can be electrically connected to the microprocessor, wherein the existing sensor detects the presence of a user within a general vicinity, which triggers the transmission of the output digital signal to the microprocessor. The microprocessor then retrieves the digital recording file stored within the computer storage to be played through the speaker system. Generally, these types of existing sensors are preferably passive infrared sensors and are conditioned to detect a presence of at least five seconds to prevent false trigger caused by someone just walking by the toilet.

According to the preferred embodiment, the device 50 further provides a method for promoting performance in a public bathroom, comprising the steps of:

detecting a user in a bathroom facility using a motion sensor, wherein detection of the user within a general vicinity triggers a transmission of an output digital signal to a microprocessor;
retrieving a digital recording file stored on a computer storage using said microprocessor upon receipt of said transmission from said motion sensor; transmitting said digital recording file to a speaker system having a digital-to-analog converter, an amplifier, and a speaker;
converting said digital recording file to an analog signal using said digital-to-analog converter;
amplifying said analog signal configured to drive said speaker using said amplifier; playing said analog signal through said speaker while the user remains within the general vicinity of said motion sensor and stopping said analog signal when the user steps away from the general vicinity of said motion sensor.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A system for promoting performance in a public restroom, comprising:
a housing storing a detection assembly therein;
said detection assembly comprising a motion sensor, a microprocessor, and a speaker system;
wherein said detection assembly is coupled to a power source;
said speaker system having a digital-to-analog converter, an amplifier, and a speaker;
wherein said microprocessor is electrically coupled to a computer and a computer storage;
said computer storage storing a digital recording file used to drive said speaker system;
said digital recording file comprising a continuous sound to alleviate stress of a user and promote bathroom performance.

2) The system for promoting performance in a public restroom of claim 1, wherein said motion sensor is a passive infrared radiation sensor configured to detect a presence of a user within a general vicinity, which triggers transmission of an output digital signal to said microprocessor and stops said transmission when said presence of the user is not within the general vicinity.

3) The system for promoting performance in a public restroom of claim 2, wherein said microprocessor, upon receipt of said output digital signal from said motion sensor, retrieves said digital recording file from said computer storage and transmits said digital recording file to said speaker system;
wherein said speaker plays said digital recording file stored on said computer storage as long as said motion sensor detects said presence of the user within the general vicinity.

4) The system for promoting performance in a public restroom of claim 1, wherein said digital recording file comprising said continuous sound includes a sound of running water.

5) The system for promoting performance in a public restroom of claim 1, wherein said power source is a battery.

6) The system for promoting performance in a public restroom of claim 1, wherein said detection assembly further comprises an on/off toggle switch configured to activate said motion sensor.

7) The system for promoting performance in a public restroom of claim 1, wherein said housing comprises at least one side and a rear surface enclosing an interior chamber;
wherein said interior chamber is configured to store said detection assembly.

8) The system for promoting performance in a public restroom of claim 7, wherein said rear surface further comprises an adhesive configured to removably mount said rear surface to a surface within a general vicinity of a bathroom toilet.

9) The system for promoting performance in a public restroom of claim 7, said side comprises a first opening designed to provide access to said on/off toggle switch, and a seconding opening designed to provide a line of sight for said motion sensor.

10) A method of promoting performance in a public bathroom, comprising the steps of:
detecting a user in a bathroom facility using a motion sensor, wherein detection of the user within a general vicinity triggers a transmission of an output digital signal to a microprocessor;
retrieving a digital recording file stored on a computer storage using said microprocessor upon receipt of said transmission from said motion sensor;
transmitting said digital recording file to a speaker system having a digital-to-analog converter, an amplifier, and a speaker;
converting said digital recording file to an analog signal using said digital-to-analog converter;
amplifying said analog signal configured to drive said speaker using said amplifier;
playing said analog signal through said speaker while the user remains within the general vicinity of said motion sensor and stopping said analog signal when the user steps away from the general vicinity of said motion sensor.
